(19) United States
(12) Patent Application Publication
(10) Pub. No.: US 2005/0039228 A1
(43) Pub. Date: Feb. 17, 2005

(54) Methods and compositions for analysis of plant gene function

(75) Inventors: Xin Shun Ding, Ardmore, OK (US); Richard S. Nelson, Ardmore, OK (US)

Correspondence Address: FULBRIGHT & JAWORSKI L.L.P., 600 CONGRESS AVE., SUITE 2400, AUSTIN, TX, 78701, US

(73) Assignee: The Samuel Roberts Noble Foundation

(21) Appl. No.: 10/871,499

(22) Filed: Jun. 18, 2004

Related U.S. Application Data
(60) Provisional application No. 60/479,905, filed on Jun. 19, 2003.
(60) Provisional application No. 60/480,705, filed on Jun. 23, 2003.

(51) Int. Cl. A01H 1/00; C12N 15/82; C12Q 1/68; C07H 21/04; C12N 5/04

(52) U.S. Cl. 800/278; 435/6; 435/419; 435/468; 536/23.6

(57) ABSTRACT

The invention provides novel methods and compositions for modulating gene function in plants. In particular, the invention provides methods and compositions that allow, for the first time, virus-induced gene silencing in rice. The invention is significant in that prior techniques were not available for rice and because of the major importance of rice to agriculture. The invention therefore provides techniques for the analysis of gene function in rice, as well as in other monocotyledonous species and dicotyledonous plant species.

### BACKGROUND OF THE INVENTION

The present invention claims benefit of priority to U.S. Provisional Ser. No. 60/479,905, filed Jun. 19, 2003, and U.S. Provisional Ser. No. 60/480,705, filed Jun. 23, 2003, the entire contents of each of these applications being hereby incorporated by reference.

1. Field of the Invention

The invention relates generally to the field of molecular biology. More specifically, it relates to compositions and methods for modulating gene function in plants.

2. Description of the Related Art

Several technologies have been used to determine plant gene function in vivo. For example, classical breeding of cultivars allows the genetic mapping of various genes. Mutagenesis of plants followed by analysis of progeny identifies gene function through loss of specific phenotypes. Transformation of plants with sequences of unknown function followed by phenotype analysis of progeny is another example of a technology used by research scientists to determine gene function. However, these techniques require a large amount of time to obtain results.

Recently, a new procedure for identifying gene function in plants has appeared and captured the interests of many plant scientists. This procedure utilizes plant viruses to express a small portion of host genes with unknown functions in the infected plant. The replication of the virus vector induces a host surveillance system that will knock out expression of genes with identity to the transiently expressed sequence through the mechanism known as virus induced gene silencing (VIGS) (van Kamman 1997; Baulcombe, 1999; Vance and Vautheret, 2001). To date, several viruses (e.g., Potato virus X, PVX, Tobacco rattle virus, TRV, Tobacco mosaic virus, TMV and Tomato golden mosaic virus, TGMV) have been successfully used as vectors for VIGS in several dicotyledonous plants (Kumagi et al., 1995; Kjemtrup et al., 1998; Ruiz et al., 1998; Burton et al., 2000; Dalmas et al., 2001; Peele et al., 2001; Ratcliff et al., 2001; Liu, 2002; Hiriart et al., 2002) and one virus, Barley stripe mosaic virus (BSMV), in a monocotyledonous plant (barley) (Holzberg et al., 2002).

VIGS occurs in plants when there is sequence similarity between the virus sequence and a plant gene sequence, either native or transgenic (Lindbo et al., 1993; Kumagai et al., 1995). It has been indicated that the mechanism involved is post-transcriptional and targets RNA molecules in a sequence-specific manner (Smith et al., 1994; Goodwin et al., 1996; Guo and Garcia, 1997). Observations that viruses can both cause and be the targets of gene silencing have suggested that the mechanism is associated with anti-viral plant defense mechanisms (Pruss et al., 1997). Gene silencing can be activated in virally infected plants when part of a gene or its RNA is perceived as part of a virus genome or transcript. This can be achieved by including a portion or all of a plant gene sequence in a viral transcript.

No virus is currently available as a vector for foreign gene expression in rice (Oryza sativa), a major crop throughout Asia and North America. There is, therefore, a great need in the art for viral vectors with broadened host ranges. Identification of a virus that infects rice and its manipulation into a vector for foreign gene expression would represent a major advance in the ability to study gene function in this important crop and potentially allow the high-throughput analysis of gene function in rice and other crops.

### SUMMARY OF THE INVENTION

Thus, in accordance with the present invention, there is provided an isolated nucleic acid sequence comprising RNA 1, RNA 2 and/or RNA 3 of F-BMV, or the complement thereof. The isolated nucleic acid sequence may have RNA 1 comprising a nucleic acid sequence selected from the group consisting of a) a nucleic acid sequence encoding the polypeptide encoded by SEQ ID NO:1; b) a nucleic acid sequence hybridizing to SEQ ID NO:1 under high stringency conditions and encoding a polypeptide having the same biological activity as the polypeptide encoded by SEQ ID NO:1; c) a nucleic acid sequence comprising the nucleic acid sequence of SEQ ID NO:1; and d) a nucleic acid sequence having 1% or less changes as compared to the nucleic acid sequence of SEQ ID NO:1. The isolated nucleic acid sequence may have RNA 2 comprising a nucleic acid sequence selected from the group consisting of a) a nucleic acid sequence encoding the polypeptide encoded by SEQ ID NO:2; b) a nucleic acid sequence hybridizing to SEQ ID NO:2 under high stringency conditions and encoding a polypeptide having the same biological activity as the polypeptide encoded by SEQ ID NO:2; c) a nucleic acid sequence comprising the nucleic acid sequence of SEQ ID NO:2; and d) a nucleic acid sequence having 1% or less changes as compared to the nucleic acid sequence of SEQ ID NO:2. “Having the same biological activity” is defined as being part of a genome that infects rice systemically at 25° C.

The isolated nucleic acid sequence may have RNA 3 comprising a nucleic acid sequence selected from the group consisting of a) a nucleic acid sequence encoding the polypeptide encoded by SEQ ID NO:3; b) a nucleic acid sequence hybridizing to SEQ ID NO:3 under high stringency conditions and encoding a polypeptide having the same biological activity as the polypeptide encoded by SEQ ID NO:3; c) a nucleic acid sequence comprising the nucleic acid sequence of SEQ ID NO:3; and d) a nucleic acid sequence having 1% or less changes as compared to the nucleic acid sequence of SEQ ID NO:3. The isolated nucleic acid sequence may have RNA 1, RNA 2 and/or RNA 3 of F-BMV comprising the corresponding sequence deposited under ATCC Accession No. PTA-5264, deposited on Jun. 13, 2003. RNA 3 may comprise specifically the nucleic acid sequence of SEQ ID NO:4. Alternatively, RNA 3 may comprise a fusion of RNA 3's from F-BMV and R-BMV.

The isolated nucleic acid sequence may further be defined as comprising the nucleic acid sequence of RNA 3 from R-BMV, or as comprising a heterologous nucleic acid sequence complementary to a target plant gene or the complement thereof. The isolated nucleic acid sequence may also further be defined as capable of replication inside a plant cell. The heterologous nucleic acid sequence may be in an untranslated region of said RNA 1, RNA 2 and/or RNA 3. The heterologous nucleic acid sequence may be in an untranslated region of RNA 3. The heterologous nucleic acid sequence is present in sense orientation, in antisense orientation, or in sense and antisense orientation. The heterologous nucleic acid sequence may comprise at least 17, 25, 50, or 100 nucleotides complementary to said target plant gene. The heterologous nucleic acid sequence may comprise a cDNA from the target plant gene. The isolated nucleic acid sequence may be further defined as RNA, DNA, single-stranded or double-stranded.

In another embodiment, there is provided a method of decreasing the expression of a plant gene in a plant cell comprising a) obtaining transcripts of RNA 1 and RNA 2 of F-BMV and RNA 3 of F-BMV or R-BMV, wherein at least one of said RNA 1, RNA 2 and/or RNA 3 further comprises a heterologous nucleic acid sequence complementary to the plant gene or the complement thereof; and b) infecting the plant cell with the transcripts. RNA 1 may comprises the nucleic acid sequence of claim 2, RNA 2 may comprise the nucleic acid sequence of claim 3, and RNA 3 may comprise the nucleic acid sequence of claim 4. The plant cell may be from a dicotyledonous plant, such as tobacco, tomato, and zucchini. The plant cell may be from a monocotyledonous plant, such as wheat, maize, rye, rice, oat, barley, turfgrass, sorghum, millet or sugarcane. The plant cell may be comprised in a plant.

In yet another embodiment, there is provided a method of identifying the function of a plant gene comprising the steps of a) obtaining transcripts of RNA 1 and RNA 2 of F-BMV and RNA 3 of F-BMV or R-BMV, wherein at least one of said RNA 1, RNA 2 and/or RNA 3 further comprises a heterologous nucleic acid sequence complementary to the plant gene or the complement thereof; b) transferring the transcripts into cells of a plant to decrease the expression of the gene; and c) identifying an altered phenotype associated with the gene based on a difference in the phenotype of cells of the plant or the whole plant relative to corresponding cells or a corresponding plant, cells of which have not taken up the transcripts. Step b) may be performed on a population of plants. Step b) may comprise transferring transcripts comprising heterologous nucleic acid sequences complementary to a plurality of plant genes, or transcripts comprising no heterologous plant genes. The plant may be a monocotyledonous plant, such as rice, or a dicotyledonous plant.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein.

FIG. 1A-D. Disease symptoms induced by F-BMV RNA transcripts. RNA transcript-inoculated C. amaranticolor (FIG. 1A) and C. quinoa (FIG. 1B) leaves showed chlorotic lesions by 4 dpi and were photographed at 6 dpi. Transcript-inoculated barley and rice plants developed systemic chlorotic streaks in young leaves by 10 dpi and were photographed at 14 (barley) (FIG. 1C) and 20 (rice) (FIG. 1D) dpi. Arrows indicate chlorotic streaks in a young barley leaf.

FIG. 2. Shows sequence of RNA 3 of R-BMV into which fragment representing partial coding sequence of the maize PDS gene was inserted (SEQ ID NO:4). The inserted PDS sequence is underlined. The construct was designated pR3-3/PDS₂₄₀.

FIG. 3A-D. Virus symptoms in leaves of different plants inoculated with the hybrid and parental BMV. FIG. 3A, systemic leaves of barley plants infected with the parental virus or hybrid BMV/PDS₂₄₀. The leaves were photographed at 15 dpi. FIG. 3B, systemic leaves of rice plants infected with the parental virus or hybrid BMV/PDS₈₆. The leaves were photographed at 21 dpi. FIG. 3C, maize plants infected or not infected (healthy) with the hybrid BMV/PDS₈₆ virus. The plants were photographed at 21 dpi. FIG. 3D, maize plants infected with parental virus or hybrid BMV/PDS₈₆ virus. The plants were photographed at 21 dpi. FIG. 3E, enlargement of a hybrid BMV/PDS₈₆ infected plant shown in C and D. Red arrows indicate chlorotic streaks induced by the parental virus. White arrows indicate light-yellow or white streaks caused by the hybrid BMV. No virus symptoms were seen in leaves of uninfected plants.

FIG. 4. Analysis of phytoene desaturase (PDS) and elongation factor 1α(EF 1α) gene expression in infected and uninfected barley leaves by RT-PCR. Young uninoculated leaves were harvested from barley plants inoculated with the parental BMV (lane 1 and 5), hybrid BMV/PDS₂₄₀ (lane 2, 3, 6 and 7) and buffer only (lane 4 and 8) at 20 dpi. Expression level of PDS gene (lane 1, 2, 3 and 4) was analyzed using primers PDS F1 and PDS R2. Expression level of EF 1α gene (lane 5, 6, 7 and 8) was analyzed using primers EF F1 and EF R1 as an internal control. PCR products were visualized in a 1.2% agrose gel. At 30 PCR cycles, PCR bands in lane 2 and 3 were weaker than those in lane 1 and 4 indicating that the expression level of the PDS gene in hybrid BMV/PDS₂₄₀ infected barley leaves was reduced.

FIG. 5A-B. Detection of the hybrid BMV/PDS₂₄₀ and parental BMV in systemically infected barley and rice leaves by immunocapture RT-PCR. FIG. 5A, barley leaves infected with the parental BMV (lane 1) and BMV/PDS₂₄₀ (lane 2 and 3) were harvested at 20 dpi. FIG. 5B, rice leaves infected with the BMV/PDS₂₄₀ (lane 1 and 2) and parental BMV (lane 3 and 4) were harvested at 18 dpi. The harvested leaves were ground in 0.1 M phosphate buffer and virion in crude extracts were captured on walls of eppendorf tubes precoated with an antibody against the R-BMV coat protein. RT-PCR was then performed using primers HKr R and B3.19F. PCR products were visualized in 1.2% agarose gels. Purified R-BMV was used as controls in both experiments (FIG. 5A, lane 4 and panel B, lane 5). DNA marker used in the experiments is the 1 kb Plus Ladder.

FIG. 6. Virus symptoms on rice plants infected with the hybrid BMV expressing or not expressing an actin gene fragment. FIG. 6A shows rice plants infected with the hybrid BMV/Actin₃₉₉. Plants infected with BMV/Actin₃₉₉ (containing a 399 bp insert of a rice actin gene) developed more severe systemic mosaic and stunting symptoms than plants infected with the parental hybrid BMV (H-BMV) lacking the actin gene insert or a mock-inoculated plant (Mock). The plants were photographed at 21 days post inoculation. FIG. 6B-D are enlargements of the leaf images boxed in FIG. 6A. FIG. 6C, a representative leaf from the hybrid BMV/actin₃₉₉ infected rice plant showing a stronger mosaic than a representative leaf from the H-BMV infected or mock-inoculated plants (FIGS. 6B and D).

FIG. 7. Actin and elongation factor 1α (EF-1α) transcript levels from plants inoculated with virus expressing or not expressing an actin gene fragment. Extracts were obtained from young systemic leaves of rice plants, similar to those shown in FIG. 6, inoculated with the parental hybrid BMV (H-BMV; lanes 1 and 2), hybrid BMV/actin₃₉₉ (H-BMV/Actin₃₉₉; lanes 3 and 4) and buffer only (Mock; lanes 5 and 6) at 21 days post inoculation. The level of the actin mRNA in each sample was analyzed using primers Actin F2 and Actin R1. EF 1α mRNA levels were analyzed using primers EF F1 and EF R1 as an internal control. PCR products obtained after 30 cycles of reaction were visualized in a 1.0% agrose gel. Each lane represents a sample from an individual independent plant. Actin transcript levels were decreased only in tissue inoculated with H-BMV containing the Actin gene insert. These results show that the BMV vector can be used to silence a range of host genes (PDS silencing results shown in FIGS. 3 and 4 and actin shown in FIGS. 6 and 7).

### DETAILED DESCRIPTION OF THE INVENTION

The invention overcomes deficiencies in the prior art by providing novel methods and compositions for the analysis of gene function in plants. In the studies of the inventors, a new isolate of brome mosaic virus (F-BMV) was identified from a naturally infected tall fescue (Festuca pratensis) plant. Analysis of host range of the virus indicated that, unlike the common strain of BMV, also known as the Russian strain of BMV, or R-BMV, F-BMV can readily infect rice, including both the Indica and Japonica varieties (Table 1). Although R-BMV has been adapted to serve as a vector for foreign gene expression in barley protoplasts (Alhquist and French, 1996; French et al., 1996), it has not been reported to operate in whole plants of rice, barley, maize and some other monocotyledonous plants. This feature of F-BMV prompted the cloning, modification and successful use of this virus by the inventors as a vector for VIGS in different plant species including rice.

The F-BMV was initially isolated from an infected tall fescue plant obtained from a breeder's stock of fescue lines and later maintained in a greenhouse at the Noble Foundation. The virus was determined to be an isolate of BMV by electron microscopy, western blot assay using an antibody against BMV coat protein and northern blot assay using an RNA profile specific for the conserved 3′ untranslated region of the BMV genomic RNAs. Because this virus infected tall fescue, it was named F-BMV.

In addition to having a broad host range, F-BMV has no known insect vector in field and is not known to be transmitted through seeds. The lack of seed transmission by BMV would make any accidental release of the modified virus vector to the environment less difficult to contain compared with other virus vectors that are seed transmitted (e.g., BSMV, PVX and TRV). Further, the ability to analyze gene function in rice is significant in that rice (Oryza sativa) is the most important crop in many countries and provides food for nearly half of the world's population. The production and availability of expressed sequence tag (EST) libraries and the availability of genomic sequences also makes rice an ideal model plant to pursue functional genomic analyses (Izawa and Shimamoto, 1996; Goffet al., 2002; Yu et al., 2002).

I. Nucleic Acids for Modulation of Plant Gene Function

The inventors have provided viral vectors for modulation of gene expression in plants. One aspect of the invention in particular provides recombinant viral vectors that may be used for silencing of one or more host genes in rice, and other monocot species, as well as some dicotyledonous plants. A representative of the vector or other nucleic acid of the current invention may, for example, be RNA and/or DNA. RNA can readily be created by in vitro transcription as described herein below. RNA may also be copied as a cDNA of a viral RNA.

Vectors provided by the invention should contain some sequences representing RNA 1 and/or RNA 2 of the F-BMV genome if the user is attempting to use the vector in rice. For other hosts it may also be important to include portions of sequence representing these RNAs for function. Such vectors also must include an RNA 3 nucleic acid from F-BMV or optionally, R-BMV instead of F-BMV or a hybrid of the two RNA 3 representatives. By including one or more nucleic acids having homology to a host gene with the foregoing vectors, gene silencing of the host gene may be achieved.

As indicated above, a modulation of the phenotype of a gene may be obtained in accordance with the invention by administering a recombinant viral nucleic acid sequence containing a second nucleic acid that has homology to a gene of interest. Such a nucleic acid may be present as a sense and/or antisense RNA and/or DNA. In order to achieve inhibition of gene expression, the added nucleic acid will generally be at least 80%, particularly at least 85%, more particularly at least 90%, and preferably at least 95% homologous in sequence to the gene of interest, or the complement thereof through at least 17, 20, 25 or 30 nucleotides of its sequence. Commonly, such sequences will hybridize to the corresponding nucleic acid sequence in the gene of interest under high stringency conditions. As used herein, “hybridization” or “hybridizes” is understood to mean the forming of a double or triple stranded molecule or a molecule with partial double or triple stranded nature. As used herein “stringent condition(s)” or “high stringency” are those conditions that allow hybridization between or within one or more nucleic acid strand(s) containing complementary sequence(s), but precludes hybridization of random sequences. For example, high stringency may be defined as 0.02M to 0.10M NaCl and 50° C. to 70° C.

It will generally be desirable that vectors provided by the invention be capable of systemic spread in an infected plant. However, such a systemic spread may not be essential for efficient gene silencing. A recombinant vector provided by the invention may or may not therefore include all cis-elements required for vascular movement of the vector or even its cell-to-cell spread. In this manner, modulation of plant gene expression in a collection of plant cells may be more efficiently carried out. Methods for inoculating plants and plant cells with recombinant viral vectors or viral particles are well known to those of skill in the art. Such vectors may, for example, be administered in a solution and may also contain any other desired ingredients including buffers, cis-elements, surfactants, solvents and similar components.

Stringent conditions tolerate little, if any, mismatch between a nucleic acid and a target strand. Such conditions are well known to those of ordinary skill in the art, and are preferred for applications requiring high selectivity. Medium stringent conditions may comprise relatively low salt and/or relatively high temperature conditions, such as provided by about 5×SSC, 50% formamide and 42° C.; or alternatively, 5×SSC, 50% formamide and 55° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture. It is also understood that compositions and conditions for hybridization are mentioned by way of non-limiting examples only, and that the desired stringency for a particular hybridization reaction in a plant cell is often determined empirically by comparison to one or more positive or negative controls. Depending on the application envisioned it is preferred to employ varying conditions of hybridization to achieve varying degrees of selectivity of a nucleic acid towards a target sequence.

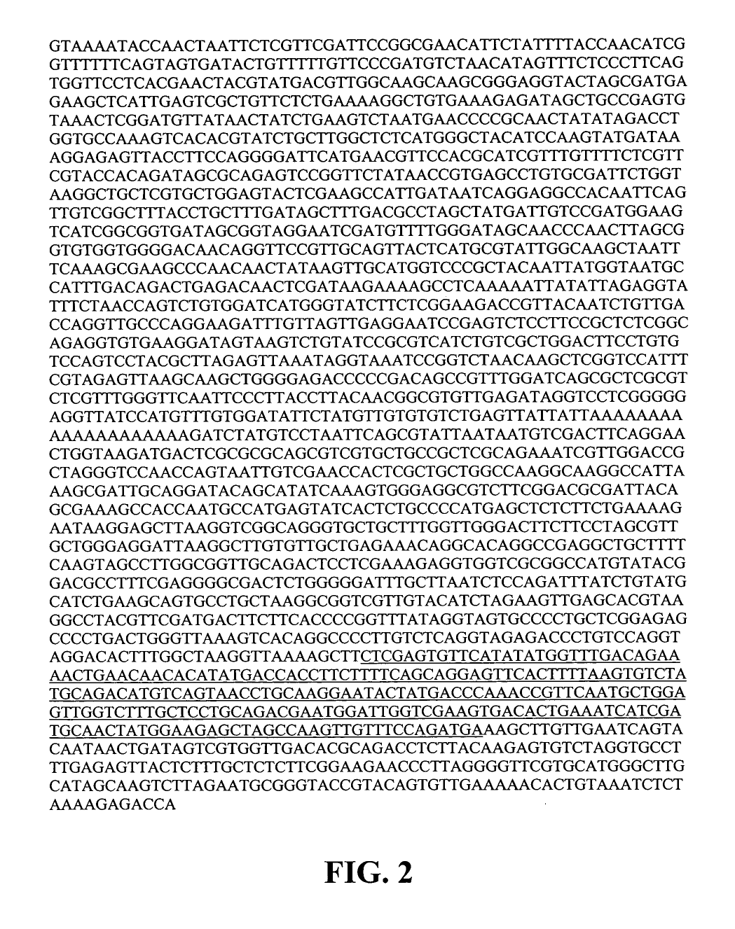

A nucleic acid sequence corresponding to a gene of interest should generally be of sufficient length that it will be unique to the coding sequence. Generally, sequence of at least 17-20 nucleotides will occur only once in most plant genomes. Benefit may also be obtained by use of longer sequences of a sense and/or antisense region of a gene of interest, including at least about 75, 100, 250 and about 500 nucleotides, including the full length of a coding region of the gene whose expression is to be reduced, as well as associated control elements. The nucleic acid may potentially be placed anywhere in an RNA 1, RNA 2 of F-BMV and/or RNA 3 or F-BMV or R-BMV. Generally, it will be preferable that the nucleic acid be placed in an untranslated region of one or more of these RNAs so that the function of the RNA or any polypeptide products translated therefrom is not adversely affected. Convenient locations for inserting such a nucleic acid are at restriction enzymes cut sites present only once in the cDNA representing the RNA. An example of such a site is shown in FIG. 2 for the cDNA representing RNA 3 of F-BMV.

Benefit may be obtained by including both sense and antisense nucleic acids for a particular gene. It will generally be preferable that the sense and antisense RNA are at least partly complementary to each other, for example, capable of secondary structures such as a stem-loop structure, which may increase the efficiency of gene silencing.

F-BMV vectors (defined as above) are useful for modulating expression of host genes in many monocots in addition to rice. Further, F-BMV also infects dicots, including cucumber, C. quinoa and Nicotiana benthamiana. The vector may thus be used to analyze gene function in a variety of plants. Examples of monocots that may be used with the invention include, but are not limited to, wheat, maize, rye, rice, oat, barley, turfgrass, sorghum, millet and sugarcane. Examples of other dicots that may be used with the invention include, but are not limited to, tobacco, tomato and zucchini.

II. Vector Construction

Construction of vectors for use with the invention will be well known to those of skill in light of the current disclosure. Recombinant constructs preferably comprise restriction endonuclease sites to facilitate vector construction. Particularly useful are unique restriction endonuclease recognition sites. Examples of such restriction sites include sites for the restriction endonucleases HindIII, Tth 1111, BsmI, KpnI and XhoI. Endonucleases preferentially break the internal phosphodiester bonds of polynucleotide chains. They may be relatively unspecific, cutting polynucleotide bonds regardless of the surrounding nucleotide sequence. However, the endonucleases which cleave only a specific nucleotide sequence are called restriction enzymes. Restriction endonucleases generally internally cleave nucleic acid molecules at specific recognition sites, making breaks within “recognition” sequences that in many, but not all, cases exhibit two-fold symmetry around a given point. Such enzymes typically create double-stranded breaks.

Many of these enzymes make a staggered cleavage, yielding DNA fragments with protruding single-stranded 5′ or 3′ termini. Such ends are said to be “sticky” or “cohesive” because they will hydrogen bond to complementary 3′ or 5′ ends. As a result, the end of any DNA fragment produced by an enzyme, such as EcoRI, can anneal with any other fragment produced by that enzyme. This properly allows splicing of foreign genes into plasmids, for example. Some restriction endonucleases that may be particularly useful with the current invention include HindIII, Tth 111 1, BsmI, KpnI and XhoI.

Some endonucleases create fragments that have blunt ends, that is, that lack any protruding single strands. An alternative way to create blunt ends is to use a restriction enzyme that leaves overhangs, but to fill in the overhangs with a polymerase, such as Klenow, thereby resulting in blunt ends. When DNA has been cleaved with restriction enzymes that cut across both strands at the same position, blunt end ligation can be used to join the fragments directly together. The advantage of this technique is that any pair of ends may be joined together, irrespective of sequence.

Those nucleases that preferentially break off terminal nucleotides are referred to as exonucleases. For example, small deletions can be produced in any DNA molecule by treatment with an exonuclease which starts from each 3′ end of the DNA and chews away single strands in a 3′ to 5′ direction, creating a population of DNA molecules with single-stranded fragments at each end, some containing terminal nucleotides. Similarly, exonucleases that digest DNA from the 5′ end or enzymes that remove nucleotides from both strands have often been used. Some exonucleases which may be particularly useful in the present invention include Bal31, S1, and ExoIII. These nucleolytic reactions can be controlled by varying the time of incubation, the temperature, and the enzyme concentration needed to make deletions. Phosphatases and kinases also may be used to control which fragments have ends which can be joined. Examples of useful phosphatases include shrimp alkaline phosphatase and calf intestinal alkaline phosphatase. An example of a useful kinase is T4 polynucleotide kinase.

Once the source DNA sequences and vector sequences have been cleaved and modified to generate appropriate ends they are incubated together with enzymes capable of mediating the ligation of the two DNA molecules. Particularly useful enzymes for this purpose include T4 ligase, E. coli ligase, or other similar enzymes. The action of these enzymes results in the sealing of the linear DNA to produce a larger DNA molecule containing the desired fragment (see, for example, U.S. Pat. Nos. 4,237,224; 4,264,731; 4,273,875; 4,322,499 and 4,336,336, which are specifically incorporated herein by reference).

It is to be understood that the termini of the linearized plasmid and the termini of the DNA fragment being inserted must be complementary or blunt in order for the ligation reaction to be successful. Suitable complementary ends can be achieved by choosing appropriate restriction endonucleases (i.e., if the fragment is produced by the same restriction endonuclease or one that generates the same overhang as that used to linearize the plasmid, then the termini of both molecules will be complementary). As discussed previously, in one embodiment of the invention, at least two classes of the vectors used in the present invention are adapted to receive the foreign oligonucleotide fragments in only one orientation. After joining the DNA segment to the vector, the resulting hybrid DNA can then be selected from among the large population of clones or libraries.

Once a DNA vector has been prepared, it will be readily understood to those of skill in the art that infective RNA transcripts may be made therefrom. For example, commercial kits are available for production of RNA transcripts. On example of such a kit that was used by the inventors is the mMeSSAGE mMACHINE transcription kit from Ambion (Austin, Tex.).

III. Assays for Gene Function or Expression

For the determination of gene function, it will generally be desired to infect a plant or part thereof with the vector carrying a sequence complementary at least in part to the gene in question at a developmental stage at which that gene will be expressed, when such information is known. For example, a phenotypic change obtained as a result of a decrease in gene expression will be most readily observed when that gene is highly expressed. A phenotypic change may be readily identified by comparison of a plant phenotype before and after being infected with a recombinant viral nucleic acid of the invention and/or by comparison with plants of a corresponding genotype which have been infected with the vector not containing the plant gene sequence or have not been infected with recombinant viral RNA.

The techniques of the invention are amenable to large-scale, high-throughput applications. For example, a plurality of recombinant vectors comprising nucleic acids homologous to a large number of plant gene(s) of unknown function could be used to infect a population of plants. In this way, the function of the corresponding gene(s) may be determined. Such plants may be infected with viral vectors at different stages of development or in different tissues depending upon the gene being assayed.

In certain embodiments of the invention, techniques may thus be used to assay gene expression and generally, the efficacy of a given gene silencing construct. While this may be carried out by visual observation of a change in plant phenotype, molecular tools may also be used. For example, expression may be evaluated by specifically identifying the nucleic acid or protein products of genes. Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Very frequently, the expression of a gene product is determined by evaluating the phenotypic results of its expression. These assays also may take many forms including but not limited to, analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may be observed, such as plant stature or growth.

III. Deposit Information

A representative deposit of F-BMV has been made with the American Type Culture Collection (ATCC), 10801 University Blvd., Manassas, Va. on Jun. 13, 2003, and given accession number PTA-5264. The deposit was made in accordance with the terms and provisions of the Budapest Treaty relating to deposit of microorganisms and was made for a term of at least thirty (30) years and at least five (05) years after the most recent request for the furnishing of a sample of the deposit is received by the depository, or for the effective term of the patent, whichever is longer, and will be replaced if it becomes non-viable during that period.

A deposit of the Russian strain of BMV was previously made by P. Alhlquist. The deposit was given ATCC accession number PV-875 (see Mise et al., 1992).

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Identification and Isolation of F-BMV

An uncharacterized virus was isolated from an infected tall fescue plant obtained from a breeder's stock of fescue lines at the University of Missouri and maintained in a greenhouse. The virus was determined to be an isolate of BMV by electron microscopy, western blot assay using an antibody against BMV coat protein and northern blot assay using an RNA profile specific for the conserved 3′ untranslated region of the BMV genomic RNAs. Because this virus infected tall fescue, it was designated F-BMV. R-BMV was from a previously described source and maintained in a growth chamber at the Noble Foundation (Ding et al., 1999).

An initial study was made of the host range of the virus. Plants of three rice cultivars were inoculated with F-BMV or R-BMV virion and grown inside a greenhouse at 25° C. The inoculated plants were observed for disease symptoms for 28 days and tested for virus infection by immunocapture RT-PCR using an antibody against the BMV coat protein and primers HK-R and B3.19F. The results indicated the ability of F-BMV to infect rice (Table 1). F-BMV was thus identified as a valuable tool and subjected to further study.

TABLE 1
Infection of rice plants with F-BMV and R-BMV

| Rice Cultivar | F-BMV | R-BMV |
|---|---|---|
| IR8 | 10/10 | 0/10 |
| PI615210 | 7/8* | 0/8 |
| Drew | 8/10 | 0/10 |

*Number of plants infected/number of plants inoculated. Two independent experiments with 4 to 5 plants per treatment were conducted.

### Example 2

### Cloning and Sequencing of F-BMV and R-BMV

Virions of F-BMV and R-BMV (Ding et al., 1999) were purified from systemically infected barley leaves by PEG precipitation and differential centrifugation (Lane, 1981). Viral RNA was then isolated from purified virions through phenol/chloroform extraction and ethanol precipitation. First-strand cDNA of RNAs 1, 2 and 3 representing each virus genome were synthesized by priming viral RNAs with Primer HK-R (5′-GACAATGGTCTCTTTTAGAG-3′) (SEQ ID NO:5). The ten 5′-most nucleotides of the HK-R primer sequence contain a PshA1 restriction site coincident with the 3′ end of the R-BMV RNA sequence. Full-length PCR products of the RNAs 1, 2 and 3 were synthesized for each virus using the HK-R primer and primers containing sequences corresponding to the T3 promoter sequence and the 5′ end of the respective R-BMV RNA sequences: (i.e., HK-1F, 5′-AATTAACCCTCACTAAAGGGAGAGTAGACCACGGAACGAGGT-3′ for RNA 1 (SEQ ID NO:6); HK-2F, 5′-AATTAACCCTCACTAAAGGGAGAGTAAACCACGGAACG-3′ (SEQ ID NO:7) for RNA 2; and HK-3F, 5′-AATTAACCCTCACTAAAGGGAGAGTAAAATACCAACT-3′ for RNA 3 (SEQ ID NO:8)).

The resulting PCR products were gel purified using QIAquick Gel Purification kit (Qiagen, Valencia, Calif.) and ligated individually into the pGEM T-Easy vector from Promega (Madison, Wis.) as instructed. Transformation of JM109 competent cells was performed as instructed by the manufacturer (Promega). Plasmid DNA was isolated from transformed cells and sequenced with specific primers. At least 3 individual full length clones were sequenced for each viral RNA component (i.e., clones representing viral RNAs 1, 2 and 3) (SEQ ID NOs:1-3). F-BMV sequences were then compared with the published sequences of R-BMV (Alhquist et al., 1984) and the BMV ATCC66 strain (Mise et al., 1994) using the DNA Star Clustal V.

### Example 3

### Production of Infectious Transcripts from cDNAs of F-BMV and R-BMV

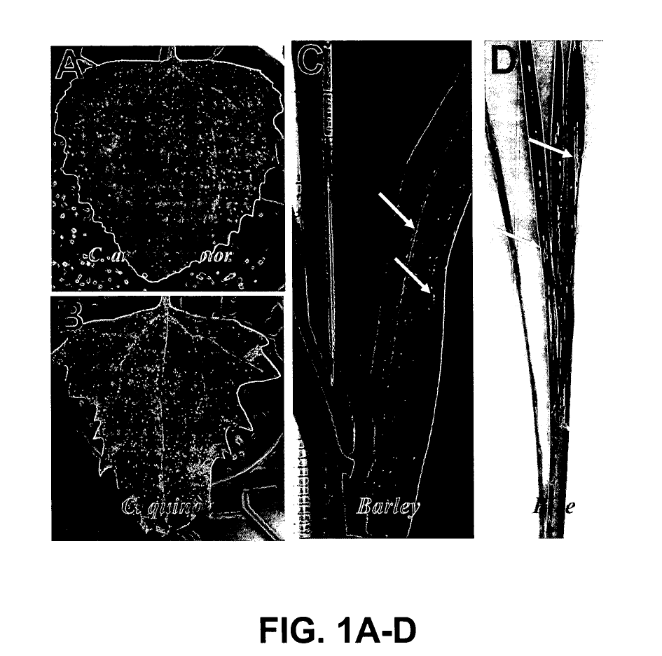

Plasmid DNAs representing RNA 1 of F-BMV (pF1-11) and R-BMV (pR1-26) were linearized with the restriction enzyme SpeI. Plasmid DNAs representing RNA 2 and 3 of the F-BMV (pF2-2 and pF3-5) and R-BMV (pR2-9 and pR3-3) were linearized with the restriction enzyme PshA. In vitro transcripts were synthesized from each clone using the mMeSSAGE mMACHINE transcription kit as described (Ambion, Austin, Tex.). The three RNA transcripts representing their respective viruses (i.e., F-BMV or R-BMV) were equally mixed and inoculated to leaves of Hordeum vulgare (barley) cv. Morex, Chenopodium amaranticolor and C. quinoa (5 to 6 μl mixed RNA transcripts/leaf, one leaf/plant). The inoculated plants were grown inside a growth chamber at 22/18° C. (day/night). At 3 to 4 days post inoculation (dpi), C. amaranticolor and C. quinoa leaves showed chlorotic lesions (FIG. 1A-1B). Barley plants inoculated with the transcripts representing F-BMV showed systemic mosaic symptoms by 7 dpi (FIG. 1C). Similar symptoms were observed on plants inoculated with the R-BMV transcripts.

Inoculation of crude extracts from F-BMV-infected barley leaves to rice cultivars IR8 and P1615210 resulted in systemic mosaic symptoms similar to those caused by the parental F-BMV (FIG. 1D). Studies using reassortant viruses indicated that RNA 1 and/or 2 of F-BMV are required for infection of rice (Table 2). For these studies, systemic leaves of rice plants were harvested at 21 dpi, ground in 0.1 M phosphate buffer and virion in crude extracts were captured on walls of eppendorf tubes precoated with an antibody against the R-BMV coat protein. RT-PCR was performed using primers HKr R and B3.19F. The results indicate that both pF3-5 and pR3-3 can be used as a vector to express foreign genes in rice plants.

TABLE 2
Reassortant BMV in Systemic Leaves of Rice Plants by
Immunocapture RT-PCR

| Rice | FFF | FFR | FRF | FRR | RFR | RFF | RRF | RRR |
|---|---|---|---|---|---|---|---|---|
| PI615210 | 10/10* | 7/10 | 4/10 | 3/10 | 5/8 | 0/10 | 0/5 | 0/10 |
| IR8 | 10/10 | 2/10 | 6/10 | 4/10 | 4/10 | 5/10 | 0/5 | 0/10 |

*Number of plants infected/number of plants inoculated with the reassortant viruses. The inoculated plants were grown inside a greenhouse at 25° C. Results are from two independent experiments.

### Example 4

### Cloning of Phytoene Desaturase (PDS) Fragments from Total RNA Isolated from Maize Leaves, Insertion Into a Viral Vector and Inoculation of Transcripts to Plants

Several fragments representing partial coding sequences of the maize PDS gene (Luo and Wurtzel, 1999) were amplified from maize leaf total RNA through RT-PCR. PCR fragments of 86 and 240 bp from the PDS gene were obtained using primers:

| | |
|---|---|
| PDS F1 | |
| (5′-CATAAGCTTCTCGAGTGTTCATATATGGTT | (SEQ ID NO:9)) |
| T-3′ and | |
| PDS R1 | |
| (5′-CATAAGCTTAGACACTTAAAAGTGAACTC- | (SEQ ID NO:10)) |
| 3′ and | |
| PDS F1 and PDS R2 | |
| (5′-CATAAGCTTTCATCTGGAAACAACTTGGC- | (SEQ ID NO:11)) |
| 3′ respectively. | |

The fragments were digested with restriction enzyme HindIII and ligated into the HindIII restriction site of the pR3-3 construct. The modified constructs (pR3-3/PDS₈₆ and pR3-3/PDS₂₄₀) were sequenced. The nucleic acid sequence of the pR3-3/PDS₂₄₀ construct is given in FIG. 2 and SEQ ID NO:4. The nucleic acid sequence of RNA 3 of the R-BMV (e.g., SEQ ID NO:4 with the PDS fragment shown FIG. 2 deleted) is given in SEQ ID NO:15). In vitro transcripts from pR3-3/PDS₈₆ or pR3-3/PDS₂₄₀ were mixed with transcripts from the pF1-11 and pF2-2 clones (referred to as hybrid BMV/PDS₈₆ and BMV/PDS₂₄₀). The mixed transcripts were inoculated to leaves of barley cv Morex C. amaranticolor and C. quinoa. The inoculated plants were grown inside a growth chamber at 22/18° C. (day/night) for 4 weeks. Transcripts from pF1-11, pF2-2 and pR3-3 not containing the PDS insert (virus referred to as the parental virus) were mixed and inoculated to plants as a control.

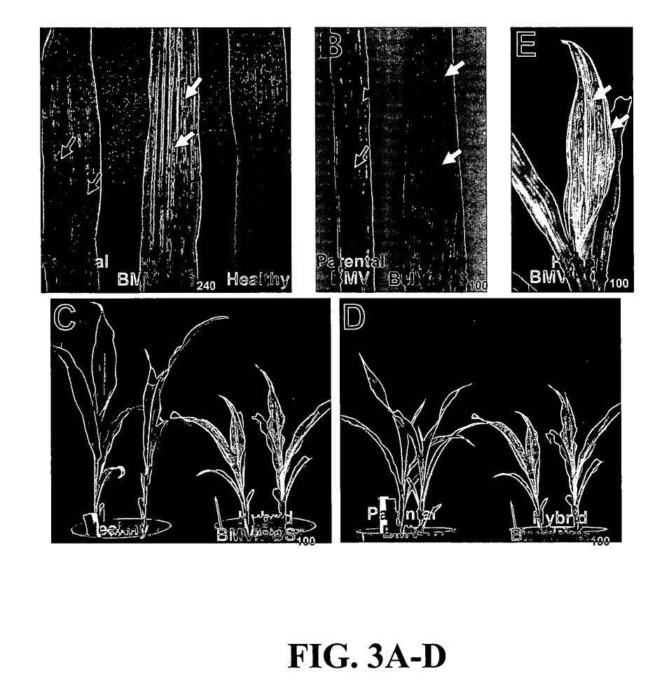

At 4 dpi, all hybrid BMV/PDS₂₄₀ virus inoculated C. amaranticolor and C. quinoa leaves showed chlorotic lesions. Chlorotic lesions were seen in the leaves by 4 dpi. The leaves were photographed at 7 dpi and the photos demonstrated chlorotic lesions in these leaves. The barley plants inoculated with the hybrid BMV/PDS₈₆ or BMV/PDS₂₄₀ developed light-yellow streaks between major veins in their young leaves (the 2^{nd} leaf above the inoculated leaf) by 10 dpi (FIG. 3A). In even younger leaves, the light-yellow streaks were also observed, but the number of streaks per leaf decreased with each succeeding leaf. This phenotype is similar to that in barley infected with BSMV harboring the PDS gene (Holzberg et al., 2002). Inoculation of crude extracts from systemically infected barley leaves showing light-yellow streaks to leaves of rice cv. 615210 and maize cv. Va35 resulted in light-yellow streaks in systemic rice leaves and white streaks in systemic maize leaves (FIG. 3B-E). Like the infected barley plants, the number of streaks on young leaves of rice and maize decreased as the plant grew.

### Example 5

### Analysis of PDS Gene Expression in Virus Infected and Uninfected Barley Leaves and the Stability of the Hybrid BMV in Systemically Infected Barley and Rice Leaves

cDNA was synthesized from total RNA isolated from systemically-infected or uninfected barley leaves using an oligodT primer. Expression of the PDS gene in barley leaves infected with the hybrid BMV and the parental virus were then determined through PCR using primers PDS F1 (SEQ ID NO:9) and PDS R2 (SEQ ID NO:11). These two primers will not amplify hybrid BMV RNA sequences. Expression levels of the elongation factor 1α (EF-1α) gene in these samples were analyzed as internal controls using primers:

| | |
|---|---|
| EF F1 | |
| (5′-GAGCATTGACAAGCGTGTGATCGAGACG- | (SEQ ID NO:12)) |
| 3′ and | |
| EF R1 | |
| (5′-CCCTCAAACCCAGAGATGGGAACGAAGGG | (SEQ ID NO:13)) |
| G-3′ | |

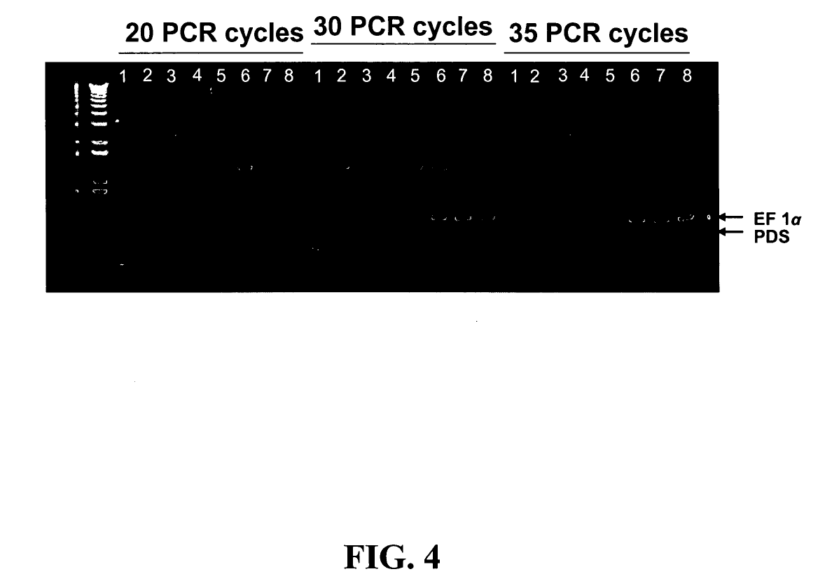

Levels of PDS gene mRNA in tissues infected with the hybrid BMV were reduced compared with that in tissues from plants infected with the parental virus or uninfected plants (FIG. 4). This result indicates that expression of the PDS sequence from the hybrid BMV/PDS₂₄₀ or BMV/PDS₈₆ genome resulted in silencing of the host PDS gene.

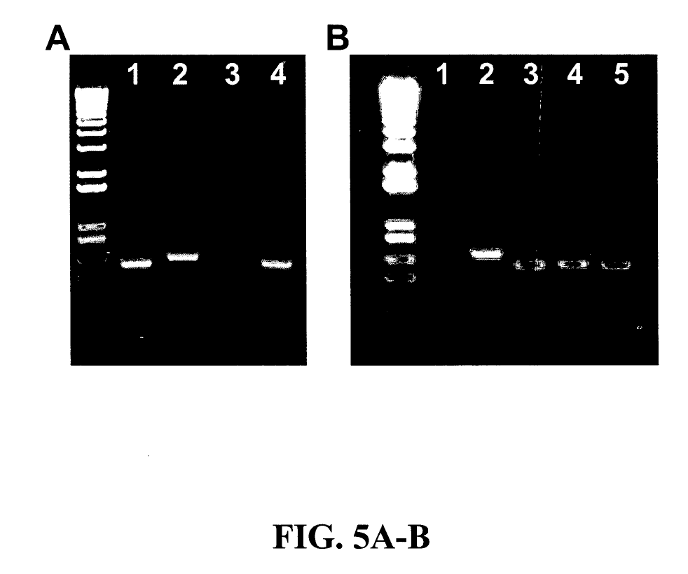

Upper young systemically-infected leaves of the infected barley plants were harvested to determine if the inserted PDS sequence was still present in the hybrid BMV genome. Young leaves were also harvested from barley and rice plants inoculated with crude extracts prepared from systemic barley leaves previously infected with the hybrid or parental BMV. Presence of the modified BMV in these tissues were confirmed by immunocapture RT-PCR using an antibody against the BMV coat protein and primers HK-R and B3.19F (5′-GTTGGGACTTCTTCCTA-3′ (SEQ ID NO:14), complementary to the nucleotides 1526-1542 of the BMV RNA 3). These two primers would amplify a partial BMV RNA 3 sequence and the inserted PDS sequence, and not the host-encoded PDS sequence. Results from these studies show that the pR3-3 construct carrying the PDS sequence can move systemically in infected barley and rice plants (FIG. 5A-B). The modified BMV construct is currently being used for gene silencing in other monocot species (e.g. tall fescue).

### Example 6

### Cloning of Rice Actin Gene Fragment into a Viral Vector and Inoculation of Transcripts to Plants and Visual Phenotypes

A rice actin gene construct was obtained from Dr. Gouliang Wang (Ohio State University). A 399 bp PCR fragment was amplified from the construct through PCR using primers Actin F3 (5′-CATAAGCTTATTATGAGCAGGAGCTGGGA-3′ (SEQ ID NO:16)) and Actin R1 (5′-CATAAGCTTTCTGCTGGAATGTGCTGAGA-3′ (SEQ ID NO:17)). The fragment was digested with restriction enzyme HindIII and ligated into the HindIII restriction site of the pR3-3 construct. The modified construct (pR3-3/actin₃₉₉) was sequenced to confirm the presence and sequence of the insertion. The nucleic acid sequence of the region is given in SEQ ID NO:19 (partial sequence of pR3-3/actin₃₉₉; sequence starts from nucleotide #1787 of pR3-3 SEQ. ID NO:15; rice actin insert is at bases 134-532 of SEQ ID NO:19). In vitro transcripts from the pR3-3/actin₃₉₉ clone were mixed with transcripts from pF1-11 and pF2-2 clones (entire viral genome referred to as hybrid BMV/actin₃₉₉; H-BMV/actin₃₉₉) and inoculated to Nicotiana benthamiana plants. The inoculated plants were grown inside a growth chamber set at 22/18° C. (day/night) and 16 hr light periods.

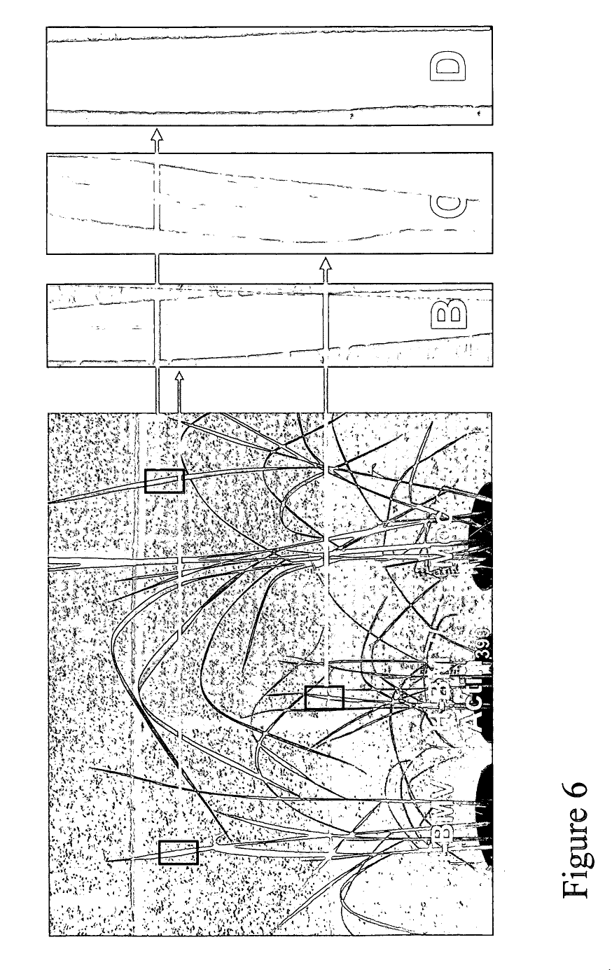

At 6 dpi, all N. benthamiana plants inoculated with H-BMV/actin₃₉₉ showed mosaic symptoms. At 12 dpi, systemic leaves of hybrid BMV/actin₃₉₉ virus infected N. benthamiana plants were harvested, ground (1:5, w/v) in phosphate buffer, pH 6.0, and inoculated to leaves of rice cv. IR8 and PI 615210. Leaf extract from N. benthamiana infected with parental H-BMV was inoculated to rice plants (cv. IR8) and analyzed as a representative of plants inoculated with virus not containing the actin insert (i.e., a virus positive control). A third set of plants was treated with inoculation buffer not containing virus (mock-inoculated). By 10 dpi, all virus inoculated plants developed systemic mosaic symptoms. By 21 dpi, stunting and chlorosis, beyond that observed in plants inoculated with H-BMV, was observed for plants inoculated with H-BMV/Actin₃₉₉ (FIG. 6). Mock-inoculated plants showed no stunting or chlorosis (FIG. 6).

### Example 7

### Analysis of Actin Gene Expression in Virus Infected and Uninfected Rice Leaves

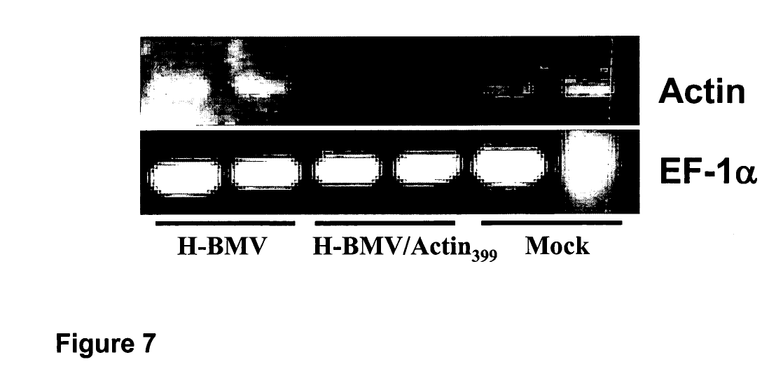

Systemic leaves were harvested from virus infected or uninfected rice cv. IR8 plants at 21 dpi. cDNA was synthesized from total RNA isolated from the harvested leaves using an oligodT primer. Expression of the actin in these leaves were then determined through PCR using primers Actin F2 (5′-CATAAGCTTTGGAGATGGTGTCAGTCACA-3′ (SEQ ID NO:18) and Actin R1 (SEQ ID NO:17)). These two primers will not amplify hybrid BMV RNA sequences. Expression level of EF 1α gene in these leaves were analyzed using primers EF F1 (SEQ ID NO:12) and EF R1 (SEQ ID NO:13) and used as internal controls. Different cycles of PCR were conducted and PCR products obtained after 30 cycles of reaction were visualized in 1.0% agrose gel and shown in FIG. 7. Results from this experiment indicate that expression of the actin gene in leaves infected with hybrid BMV/actin₃₉₉ virus was reduced and thus silenced through VIGS.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The references listed below are incorporated herein by reference to the extent that they supplement, explain, provide a background for, or teach methodology, techniques, and/or compositions employed herein.
Abdullah et al, Biotechnology, 4:1087, 1986.
Araki et al., J. Mol. Biol. 225(1):25-37, 1992.
Bansal et al., Proc. Natl. Acad. Sci. USA, 89:3654-3658, 1992.
Bates, Mol. Biotechnol., 2(2):135-145, 1994.
Battraw and Hall, Theor. App. Genet., 82(2):161-168, 1991.
Bhattacharjee and Gupta, J Plant Bioch. and Biotech. 6:(2):69-73, 1997.
Boisson et al., Mol. Plant Microbe Interactions, 14:695-700, 2001.
Bolivar et al., Gene, 2: 95-113, 1977.
Bower et al., Plant Journal, 2:409-416, 1992.
Buchanan-Wollaston et al., Plant Cell Reports, 11:627-631, 1992.
Buising and Benbow, Mol. Gen. Genet., 243(1):71-81, 1994.
Callis et al., Genes Dev., 1: 1183-1200, 1987.
Casa et al., Proc. Natl. Acad. Sci. USA, 90(23):11212-11216, 1993.
Cashmore et al., Gen. Eng. of Plants, Plenum Press, NY, 29-38, 1983.
Chandler et al., Plant Cell, 1:1175-1183, 1989.
Christensen, and Quail, Transgenic Research, 5(3):213-218., 1996.
Christou et al., Proc. Natl. Acad. Sci. USA, 84(12):3962-3966, 1987.
Clough and Bent, Plant J., 16, 735-743, 1998.
Conkling et al., Plant Physiol., 93:1203-1211, 1990.
Cordero et al., Plant J, 6(2)141-150, 1994.
DE 3642 829 A
De Block et al., EMBO J, 6(9):2513-2518, 1987.
De Block et al., Plant Physiol., 91:694-701, 1989.
Dennis et al., Nucl. Acids Res., 12(9):3983-4000, 1984.
D'Halluin et al., Plant Cell, 4(12):1495-1505, 1992.
Ebert et al., Proc. Natl. Acad. Sci. USA, 84:5745-5749, 1987.
Enomoto, et al., J. Bacteriol., 6(2):663-668, 1983.
European Appl. 154,204
Fraley et al., Bio/Technology, 3:629-635, 1985.
Franken et al., EMBO J, 10(9):2605-2612, 1991.
Fromm et al., Nature 319:791-793, 1986.
Gelvin et al., In: Plant Molecular Biology Manual, 1990.
Ghosh-Biswas et al., J Biotechnol., 32(1):1-10, 1994.
Golic and Lindquist, Cell, 59:3, 499-509, 1989.
Goodwin et al, Plant Cell 8, 95-105, 1996.
Guilley et al., Cell, 30:763, 1982.
Guo and Garcia, Mol. Plant-Microbe Interact. 10, 160-170, 1997.
Hagio et al., Plant Cell Rep., 10(5) 260-264, 1991.
Haseloff et al., Proc. Natl. Acad. Sci. USA, 94(6):2122-2127, 1997.
He et al., Plant Cell Reports, 14 (2-3):192-196, 1994.
Hensgens et al., Plant Mol. Biol., 22(6):1101-1127, 1993.
Hiei et al., Plant. Mol. Biol., 35(1-2):205-218, 1997.
Hou and Lin, Plant Physiology, 111:166, 1996.
Hudspeth and Grula, Plant Mol. Biol., 12:579-589, 1989.
Ishida et al., Nat. Biotechnol., 14(6):745-750, 1996.
Jefferson, Plant Mol. Biol. Rep., 5:387-405, 1987.
Kaeppler et al., Plant Cell Reports 9: 415-418, 1990.
Keller et al., EMBO J, 8(5):1309-1314, 1989.
Klee et al., Bio-Technology, 3(7):637-642, 1985.
Knittel et al., Plant Cell Reports, 14(2-3):81-86, 1994.
Kohler et al., Plant Mol. Biol., 29(6):1293-1298, 1995.
Kumagai et al, Proc. Natl. Acad. Sci. USA 92, 1679-1 683, 1995.
Kunkel et al., Methods Enzymol., 154:367-382, 1987.
Langridge et al., Proc. Natl. Acad. Sci. USA, 86:3219-3223, 1989.
Lawton et al., Plant Mol. Biol., 9:315-324, 1987.
Lazzeri, Methods Mol. Biol., 49:95-106, 1995.
Lee et al., Korean J Genet., 11(2):65-72, 1989.
Lindbo et al, Plant Cell 5, 1749-1 759, 1993.
Lindstrom et al., Developmental Genetics, 11:160, 1990.
Lorz et al., Mol Gen Genet., 199:178-182, 1985.
Maeser et al, Mol. Gen. Genet., 230(1-2):170-176, 1991.
Marcotte et al., Nature, 335:454, 1988.
Martinez et al., J. Mol. Biol., 208(4):551-565, 1989.
McCabe and Martinell, Bio-Technology, 11 (5):596-598, 1993.
McCormac et al., Euphytica, 99(1):17-25, 1998.
Mise K, et al. Nucleotide sequence responsible for the synthesis of a truncated coat protein of brome mosaic virus strain ATCC66. J. Gen. Virol. 73: 2543-2551, 1992.
Murakami et al., Mol. Gen. Genet., 205:42-50, 1986.
Mylona et al., Plant Mol. Biol., 26:39-50, 1994.
Nagatani et al., Biotech. Tech., 11(7):471-473, 1997.
Odell et al., Nature, 313:810-812, 1985.
Ogawa et al., Sci. Rep., 13:42-48, 1973.
Omirulleh et al., Plant Mol. Biol., 21(3):415-428, 1993.
Ow et al., Science, 234:856-859, 1986.
PCT Appl. WO 9217598
PCT Appl. WO 94/09699
PCT Appl. WO 95/06128
PCT Appl. WO 95/06128
PCT Appl. WO 97/4103
PCT Appl. WO 97/41228
Poszkowski et al., EMBO J, 3:2719, 1989.
Potrykus et al., Mol. Gen. Genet., 199:183-188, 1985.
Poulsen et al., Mol. Gen. Genet., 205(2):193-200, 1986.
Pruss et al, Plant Cell, 859-868, 1997.
Quigley et al., J. Mol. Evol., 29(5):412-421, 1989.
Ralston et al., Genet., 119(1):185-197, 1988.
Reichel et al., Proc. Natl. Acad. Sci. USA, 93(12):5888-5893, 1996.
Rhodes et al., Methods Mol. Biol., 55:121-131, 1995.
Ritala et al., Plant Mol. Biol., 24(2):317-325, 1994.
Rochester et al., EMBO J, 5:451-458, 1986.
Rogers et al., Methods Enzymol., 153:253-277, 1987.
Sambrook et al., In Molecular Cloning: A Laboratory Manual, Second edition, Cold Spring Harbor Laboratory, Cold Spring Harbor, N.Y., 1989.
Sauer, Mol. and Cell. Biol., 7:2087-2096, 1987.
Schwob et al., Plant J, 4(3):423-432, 1993.
Sheen et al., Plant J, 8(5):777-784, 1995.
Simpson, Science, 233:34, 1986.
Singsit et al., Transgenic Res., 6(2):169-176, 1997.
Smith et al, Plant Cell 6, 1441-1453, 1994.
Spencer et al., Plant Molecular Biology, 18:201-210, 1992.
Stalker et al., Science, 242:419-422, 1988.
Sullivan et al., Mol. Gen. Genet., 215(3):431-440, 1989.
Sutcliffe, Proc. Natl. Acad. Sci. USA, 75:3737-3741, 1978.
Thillet et al., J. Biol. Chem., 263:12500-12508, 1988.
Thompson et al., EMBO J, 6(9):2519-2523, 1987.
Thompson et al., Euphytica, 85(1-3):75-80, 1995.
Tian et al., Plant Cell Rep., 16:267-271, 1997.
Tingay et al., Plant J, 11(6):1369-1376, 1997.
Tomes et al., Plant. Mol. Biol. 14(2):261-268, 1990.
Tomic et al., Nucl. Acids Res., 12:1656, 1990.
Torbet et al., Crop Science, 38(1):226-231, 1998.
Torbet et al., Plant Cell Reports, 14(10):635-640, 1995.
Toriyama et al., Theor Appl. Genet., 73:16, 1986.
Tsukada et al., Plant Cell Physiol., 30(4)599-604, 1989.
U.S. Pat. No. 4,237,224
U.S. Pat. No. 4,264,731
U.S. Pat. No. 4,273,875
U.S. Pat. No. 4,322,499
U.S. Pat. No. 4,336,336
U.S. Pat. No. 4,535,060
U.S. Pat. No. 5,302,523
U.S. Pat. No. 5,322,783
U.S. Pat. No. 5,384,253
U.S. Pat. No. 5,464,765
U.S. Pat. No. 5,508,184
U.S. Pat. No. 5,508,468
U.S. Pat. No. 5,527,695,
U.S. Pat. No. 5,538,877
U.S. Pat. No. 5,538,880
U.S. Pat. No. 5,545,818
U.S. Pat. No. 5,550,318
U.S. Pat. No. 5,563,055
U.S. Pat. No. 5,591,616
U.S. Pat. No. 5,610,042
U.S. Pat. No. 5,641,876
U.S. Pat. No. 5,658,772
Uchimiya et al., Mol. Gen. Genet., 204:204, 1986.
Upender et al., Biotechniques 18(1):29-30, 1995.
Van Eck et al., Plant Cell Reports, 14(5):299-304, 1995.
Van Tunen et al, EMBO J, 7:1257, 1988.
Vasil et al., Plant Physiol., 91:1575-1579, 1989.
Vodkin et al., Cell, 34:1023, 1983.
Vogel et al., J. Cell. Biochem., (Suppl. 0) 13:Part D, 1989.
Walker et al., Proc. Natl. Acad. Sci. USA, 84:6624-6628, 1987.
Wang et al., Mol. Cell. Biol., 12(8):3399-3406, 1992.
Watson and Ramstad, Corn: Chemistry and Technology, 1987.
Wenzler et al., Plant Mol. Biol., 12:41-50, 1989.
Yarnada et al., Plant Cell Rep., 4:85, 1986.
Yang and Russell, Proc. Natl. Acad. Sci. USA, 87:4144-4148, 1990.
Zheng and Edwards, J. Gen. Virol., 71:1865-1868, 1990.
Zhou et al., Plant Cell Reports, 12(11).612-616, 1993.

1. An isolated nucleic acid sequence comprising RNA 1, RNA 2 and/or RNA 3 of F-BMV, or the complement thereof.

2. The isolated nucleic acid sequence of claim 1, wherein said RNA 1 comprises a nucleic acid sequence selected from the group consisting of:
a) a nucleic acid sequence encoding the polypeptide encoded by SEQ ID NO:1;
b) a nucleic acid sequence hybridizing to SEQ ID NO:1 under high stringency conditions, and encoding a polypeptide and/or RNA having the same biological activity as the polypeptide encoded by SEQ ID NO:1 and/or RNA 1;
c) a nucleic acid sequence comprising the nucleic acid sequence of SEQ ID NO:1; and
d) a nucleic acid sequence having 1% or less changes as compared to the nucleic acid sequence of SEQ ID NO:1.

3. The isolated nucleic acid sequence of claim 1, wherein said RNA 2 comprises a nucleic acid sequence selected from the group consisting of:
a) a nucleic acid sequence encoding the polypeptide encoded by SEQ ID NO:2;
b) a nucleic acid sequence hybridizing to SEQ ID NO:2 under high stringency conditions, and encoding a polypeptide and/or RNA having the same biological activity as the polypeptide encoded by SEQ ID NO:2 and/or RNA 2;
c) a nucleic acid sequence comprising the nucleic acid sequence of SEQ ID NO:2; and
d) a nucleic acid sequence having 1% or less changes as compared to the nucleic acid sequence of SEQ ID NO:2.

4. The isolated nucleic acid sequence of claim 1, wherein said RNA 3 comprises a nucleic acid sequence selected from the group consisting of:
a) a nucleic acid sequence encoding the polypeptide encoded by SEQ ID NO:3;
b) a nucleic acid sequence hybridizing to SEQ ID NO:3 under high stringency conditions, and encoding a polypeptide and/or RNA having the same biological activity as the polypeptide encoded by SEQ ID NO:3 and/or RNA 3;
c) a nucleic acid sequence comprising the nucleic acid sequence of SEQ ID NO:3;
d) a nucleic acid sequence having 1% or less changes as compared to the nucleic acid sequence of SEQ ID NO:3.

5. The isolated nucleic acid sequence of claim 1, wherein said RNA 1, RNA 2 and/or RNA 3 of F-BMV comprise the corresponding sequence deposited under ATCC Accession No. PTA-5264.

6. The isolated nucleic acid sequence of claim 1, further defined as comprising the nucleic acid sequence of RNA 3 from R-BMV.

7. The isolated nucleic acid sequence of claim 6, wherein said RNA 3 comprises the nucleic acid sequence of SEQ ID NO:15.

8. The isolated nucleic acid sequence of claim 1, further defined as comprising a heterologous nucleic acid sequence complementary to a target plant gene or the complement thereof.

9. The isolated nucleic acid sequence of claim 8, wherein the heterologous nucleic acid sequence is in an untranslated region of said RNA 1, RNA 2 and/or RNA 3.

10. The isolated nucleic acid sequence of claim 1, wherein the heterologous nucleic acid sequence is in an untranslated region of RNA 3.

11. The isolated nucleic acid sequence of claim 6, wherein RNA 3 comprises the nucleic acid sequence of SEQ ID NO:4.

12. The isolated nucleic acid sequence of claim 8, wherein the heterologous nucleic acid sequence is present in sense orientation.

13. The isolated nucleic acid sequence of claim 8, wherein the heterologous nucleic acid sequence is present in antisense orientation.

14. The isolated nucleic acid sequence of claim 8, wherein the heterologous nucleic acid sequence is present in sense and antisense orientation.

15. The isolated nucleic acid sequence of claim 8, wherein the heterologous nucleic acid sequence comprises at least 17 nucleotides complementary to said target plant gene.

16. The isolated nucleic acid sequence of claim 8, wherein the heterologous nucleic acid sequence comprises at least 25 nucleotides complementary to said target plant gene.

17. The isolated nucleic acid sequence of claim 8, wherein the heterologous nucleic acid sequence comprises at least 50 nucleotides complementary to said target plant gene.

18. The isolated nucleic acid sequence of claim 8, wherein the heterologous nucleic acid sequence comprises at least 100 nucleotides complementary to said target plant gene.

19. The isolated nucleic acid sequence of claim 8, wherein the heterologous nucleic acid sequence comprises a cDNA from the target plant gene.

20. The isolated nucleic acid sequence of claim 1, further defined as RNA.

21. The isolated nucleic acid sequence of claim 1, further defined as DNA.

22. The isolated nucleic acid sequence of claim 1, further defined as single-stranded.

23. The isolated nucleic acid sequence of claim 1, further defined as double-stranded.

24. The isolated nucleic acid sequence of claim 1, wherein RNA 3 comprises a fusion of RNA 3's from F-BMV and R-BMV.

25. The isolated nucleic acid sequence of claim 1, further defined as capable of replication inside a plant cell.

26. A method of decreasing the expression of a plant gene in a plant cell comprising:
a) obtaining transcripts of RNA 1 and RNA 2 of F-BMV and RNA 3 of F-BMV or R-BMV, wherein at least one of said RNA 1, RNA 2 and/or RNA 3 further comprises a heterologous nucleic acid sequence complementary to the plant gene or the complement thereof; and
b) infecting the plant cell with the transcripts.

27. The method of claim 26, wherein RNA 1 comprises the nucleic acid sequence of claim 2.

28. The method of claim 26, wherein RNA 2 comprises the nucleic acid sequence of claim 3.

29. The method of claim 26, wherein RNA 3 comprises the nucleic acid sequence of claim 4.

30. The method of claim 26, wherein the plant cell is from a dicotyledonous plant.

31. The method of claim 30, wherein the dicotyledonous plant is tobacco, tomato, and zucchini.

32. The method of claim 26, wherein the plant cell is from a monocotyledonous plant.

33. The plant of claim 32, wherein the monocotyledonous plant is wheat, maize, rye, rice, oat, barley, turfgrass, sorghum, millet or sugarcane.

34. The method of claim 26, wherein the plant cell is comprised in a plant.

35. A method of identifying the function of a plant gene comprising the steps of:
a) obtaining transcripts of RNA 1 and RNA 2 of F-BMV and RNA 3 of F-BMV or R-BMV, wherein at least one of said RNA 1, RNA 2 and/or RNA 3 further comprises a heterologous nucleic acid sequence complementary to the plant gene or the complement thereof;
b) transferring the transcripts into cells of a plant to decrease the expression of the gene; and
c) identifying an altered phenotype associated with the gene based on a difference in the phenotype of cells of the plant or the whole plant relative to corresponding cells or a corresponding plant, cells of which have not taken up the transcripts.

36. The method of claim 35, wherein step b) is performed on a population of plants.

37. The method of claim 36, wherein step b) comprises transferring transcripts comprising heterologous nucleic acid sequences complementary to a plurality of plant genes, or transcripts comprising no heterologous plant genes.

38. The method of claim 35, wherein the plant is a monocotyledonous plant.

39. The method of claim 38, wherein the plant is rice.

40. The method of claim 35, wherein the plant is a dicotyledonous plant.


SEQUENCE LISTING (19 sequences)

SEQ ID NO: 1 - DNA, 3234 bp, Artificial Sequence
  Description of Artificial Sequence  Synthetic Primer
gtagaccacggaacgaggttcaatcccttgtcgaccacggttctactacttgttctttgtttttcaccaacaaaatgtcaagttctatcgatttgctgaagttgattgctgagaagggtgctgccagccagagtgcccaagacatcgtagacaatcaggtcgcgcaacagttatctgcgcagattgaatacgcgaaaaggtctaagaaaatcaacgttcgcaataagctctctattgaggaggctgacgccttccgtgaccgttatggtggtgcctttgatttaaatttgactcagcagtatcatgcaccccatagcctggctggtgctctgcgtgtagcggagcattacgactgtctcgacagttttccccctgaagaccccgttatagatttcggagggtcttggtggcatcacttttcaagaagggataaaagggtgcacagttgttgtcctgtgttgggtgttagagacgctgcccgacatgaggagaggatgtgccgcatgcggaaaattttgcaagaaagcgatgatttcgatgaagtcccgaacttttgtcttaaccgagctcaagattgtgatgtccaagctgattgggctatctgtatccatggcggttatgatatgggcttccaaggtctgtgtgacgccatgcattcgcatggagtacgcgtactacgtggtaccgttatgttcgacggcgccatgttgtttgaccgcgagggttttcttcccttgcttaaatgtcactggcaacgtgacgggtcaggcgcggatgaggtgatcaaattcgactttgaaaatgaaagcacattatcttacatccatggatggcaagatttgggctcctttttcaccgagtcggtgcattgcatcgatggaaccacctatctgttggagcgcgaaatgctgaaatgtaacatcatgacctataagatcatcgctacaaatttacgctgcccccgggagacactacgtcactgtgtatggtttgaagacatatctaagtacgtgggagtctcaatacctgaagactggagtctcaatcgctggaaatgtgtgcgcgtcgccaaaaccacagtgagagaggtagaggagatagctttcagatgtttcaaggaaaataaagaatggaccgagaacatgaaagctgtcgcatctatcttatccgccaagtcgtcgactgttattattaacggtcaggctatcatggctggtgagcgcttagacattgaagattatcatctagtggcctttgctttgactttgaatctgtatcaaaagtacgaaaagcttacggccctccgcgatgggatggaatggaaaggttggtgccatcacttcaaaactaggttttggtggggtggagattcatccagggcgaaagtaggatggctgagaacattggctagcagatttcccctactacgtctggattcttatgcggacagttttaagtttctgactcgtctctcaaacgttgaagaatttgagcaagattctgtaccaatatcacgtttgagaacgttttggactgaagaggacttattcgaccggctggagcatgaagtgcagacagccaagaccaagcgctcgaagaagaaggcgatagtcccgccagctgctgagatacctcaggaggagtttcatgatgcccctgagagttcgagccctgagtccgtcagtgatgacgttaaaccggtgactgatgtggtcccggatgccgaggtgtctgttgaggtaccaacggaccctcgtggcatatctagacacggagccatgaaggaatttgtgcgttattgtaagagattacataacaactccgagtctaatcttcgtcacctatgggacatttccggcggtcgcggaagtgagatcgcaaataagagcatcttcgagacctaccatcgcatagacgatatggtgaatgtccatttggccaacggtaactggttgtatcctaaaaaatacgattacaccgttggatataatgagcatggtttaggtccgaagcacgcagatgaaacgtacattgttgataaaacatgtgcatgctctaacttgagggacattgcagaagctagcgccaaagtttctgtccctacatgcgatatttccatggttgatggagttgcgggatgcggtaaaaccactgccataaaagatgcattccgtatgggagaggacctaattgtgacggcgaatcgtaaatcggccgaggacgtcaggatggctttattccctgacacttttaattccaaggtagctttggatgttgtgcgcaccgcggattctgcgatcatgcacggtgtaccgtcctgtcataggctgcttgttgatgaggctggtttactacattacggtcaactcctggtggtggctgctctgtctaaatgttcacaagttcttgcctttggggacacagagcagatttcgttcaagtctcgtgacgcgggttttaaattgctccacggtaatctgaaatatgatcgccgtgacgttgttcacaagacttaccggtgtccgcaagatgttatcgctgctgttaatctgctgaagcgtaaatgcggtaatagggacacgaagtatcaatcctggacatctgagtccaaagtttctagaagtctcacgaagcgtcgtattacttctgttttgcaggtcactattgatccgaacagaacgtatcttacgatgactcaagctgataaagcggcccttcaaacgagggctaaggactttcccgtgagcaaggactggattgatggacatataaaaacagtacacgaagcgcaagggatctctgttgacaatgtcgctttggttcggcttaagtcgaccaaatgtgatttgtttaaacatgaggagtactgtttggttgccttaacacgacacaagaagtcctttgagtattgctttaacggcgagctcgctggtgatttgatctttaattgtgttaagtgatgcgcttgtctctgtgtgagacctctgctcgaggagagccctgttccaggtaggaacgttgtggtctaacttaagactagctgaatcggtgctataaccgatagtcgtggttgacacgcagacctcttacaagagtgtctaggcgcctttgagagttactctttgctctcttcggaagaacccctaggggttcgtgcatgggcttgcatagcaagtcttagaatgcgggtgccttacagtgttgaaaaacactgtaaatctctaaaagagacca

SEQ ID NO: 2 - DNA, 2867 bp, Artificial Sequence
  Description of Artificial Sequence  Synthetic Primer
gtaaaccacggaacgaggttcaatcccttgtcgacccacggtttgcgcaacacacatctaaccttgttgttgttgtgttttgtttctttctactatcaccaagatgtcttcgaaaacctgggatgatgatttcgttcgccaggtcccgtctttccaatggatcatagatcaatccttagaagacgaggtggaggctgctagccttcaggtgcaggagccggcagacggagttgccattgacggatctctcgcgagttttaaattagctatagcgcccttggagataggaggggtgttcgatcccccttttgaccgagtgcgctggggctctatttgcgataccgtccagcaaatggttcaacagttcaccgatagaccactgattcctcaggctgaaatggcacggatgttatatcttgacattccgggctcttttgtgctcgaagatgaaatcgatgactggtatcctgaggatactagtgacggttacggtgtatcgtttgccgccgatgaagatcatgcgagcgatctaaaactcgccagtgattcctcgaactgtgaaattgaggaagttcgtgttactggagatacccccaaggagctgacccttggagataggtacatgggcattgacgaagagtttcagactaccaatactgattacgacatcactcttcagatcatgaatcctattgaacatagggtttcgcgcgttattgatacgcactgccatccagataaccctgacatctccactgggccaatttatatggagagagtcagccttgctagaacagaagcaaccagtcattccatactgccaacccatgcttatttcgatgattcgtaccatcaagcccttgttgagaatggtgattattccatggactttgataggatcagacttaagcaaagtgatgtagactggtacagggaccccgataaatattttcaaccaaaaatgaatatcgggagtgctcagcgaagagttggtactcagaaagaagtcttaaccgcacttaaaaagcgaaacgcggatgttccagaaatgggagacgcgattaacatgaaggaaactgcgaaggctatagcaaagcgctttcgtagcacattccttaatgttgacggtgaagactgtctgagagcttctatggatgtcatgactaagtgtcttgagtaccataagaagtggggtaagcacatggacttacaaggtgtgaatgtggcagcagagactgatttacgtcggtaccagcatatgctgaagtctgatgtaaaacctgttgtaactgacacccttcacttggaacgagcagtagcagctactataacatttcatagtaaaggtgtgactagtaatttttcaccctttttcactgcttgtttcgagaagttatcattggccctgaaatccaggttcattgtgcctatcggaaagatatcctctctggagcttaagaatgtccgcttgaataacagatactttcttgaagcggacctaagcaaatttgataaatctcagggtgagctgcacctagagtttcagagagagatacttcttgcgctgggctttccagcgccgctgacgaattggtggtctgattttcatcgcgattcttatttatcagaccctcatgccaaggtgggaatgtccgtttccttccaacgcagaactggtgatgcgtttacatatttcgggaatactcttgtcactatggctatgattgcatatgcctctgatctaagtgactgtgactgtgcaatattctcaggagatgattctttaatcatttctaaagttaaaccagtcttggataccgatatgttcacgtctctctttaatatggagataaaagttatggaccctagtgtgccctacgtttgtagtaagtttcttgttgaaactgaaatgggcaattgggtgtctataccagaccctatgagagagatccagcgcttagctaagcgaaagattctgcgtgaagaacagatgctcagagcacatttcgtttccttctgcgatcgaatgaagtttattaatcaacttgatgagaagatgattacgatgctctgtcattttgtttatctgaaatatgggaaagaaaaaccttggattttcgaggaggttagagctgctcttgcggctttttctttatactccgagaatttcctgaggttctctgattgctactgtaccgaaggcatcagagtttatcagatgagcgatcctgtatgtaagttcaaacgcactatggaagagcgtaaaactgatggtgactggtttcacaactggaagaatccaaagtttcctggtgtgttagacaaagtctacagaacccttggaatttattcctcggactgtagtactaaggagctccctgtcaaacggatcggacgtttacatgaggcccttgagcgtgagtcactcagattagcgaatgatcgtaggaccacacaacgcttgaaaaagaaggtcgacgattacgctaccggtagaggaggcctaacgtcagttgatgctttgctcttgaagtcccattgtgagacttttaagccctctgatctgagatgatcggttctatatgatatatgaacctaagctgtgagcagccctttggttaaggttaaaaactcctggtcaggtagaccactttggctaagtttaaaagcttgttgaatcagtacaataactgatagtcgtggttgacacgcagacctcttacaagagtgtctaggtgcctttgagagttactctttgctctcttcggaagaacccttaggggttcgtgcatgggcttgcatagcaagtcttagaatgcgggtaccgtacagtgttgaaaaacactgtaaatctctaaaagagacca

SEQ ID NO: 3 - DNA, 2114 bp, Artificial Sequence
  Description of Artificial Sequence  Synthetic Primer
gtaaaataccaactaattctcgttcgattccggcgaacattctattttaccaacatcggttttttcagtagtgatactgtttttgttcccgatgtctaacatagtttctcccttcagtggttcctcacgaactacgtctgacgttggcaagcaagcgggaggtactagcgacgagaagctcattgagtcgctgttctctgaaaaggctgtgaaagagatagctgccgagtgtaaactcggatgttataactatctgaagtctaatgaaccccgcaactatatagacctggtgccaaagtcacacgtatctgcttggctctcatgggctacacccaagtatgataaaggagagttaccttccaggggattcatgaacgttccacgcatcgtttgttttctcgttcgtaccacagatagcgcagagtccggttctataaccgtgagcctgtgcgattctggtaaggctgctcgtgctggagtactcgaagccattgataatcaggaggccacaattcagttgtcggctttacctgctttgatagctttgacgcctagctatgattgtccgatggaagtcatcggcggtgatagcggtaggaatcgatgttttgggatagcaacccaacttagcggtgtggtggggacaacaggttccgttgcagttactcatgcttattggcaagctaatttcaaagcgaagcccaacaactataagttgcatggtcccgctacaattatggtaatgccatttgacagactgagacaactcgataagaaaagcctcaaaaattacatcagaggtatttctaaccagtctgtggatcatgggtatcttctcggaagaccgttacaatctgttgatcaggttgcccaggaagatttgttagttgaggaatccgagtctccttccgctctcggcagaggtgtgaaggatagtaagtctgtatccgcgtcatctgtcgctggacttcctgtgtccagtccttcgcttagaattaaataggtaaatccggtctaacaagcttggtccatttcgtggagttaagcaagctggggagacccccgacagccgtttggatcagcgctcgcgtctcgtttgggttcaattcccttaccttacaacggcgtgttgagataggtcctcgggggaggttatccatgtttgtggatattctatgttgtgtgtctgagttattaaaaaaaaaaaaaaaaaaaaaagatctatgtcctaattcagcgtattaataatgtcgacttcaggaactggtaagatgactcgcgcgcagcgtcgagctgccgctcgtagaaatcgtcggaccgctacggtccaaccggtaattgtcgaaccattcgctgctggccaaggcaaggccattaaagcgattgcaggatacagcatatcaaagtgggaggcgtcttcggacgcgattacagcgaaagccaccaatgccatgagtatcactctgccccatgagctctcttctgaaaagaataaggagcttaaggtcggcagggtgctgctttggttgggacttcttcctagcgttgctgggaggattaaggcttgtgttgctgagaaacaggcacaggccgaggccgcttttcaagtagccttggcggttgctgactcctcgaaagaggtggtcgcggccatgtatacggacgcctttcgaggggcgactctgggggatttgcttaatctccagatttatctgtatgcatctgaagcagtgcctgctaaggcggtcgttgtacatctagaagttgagcacgtaaggcctacgttcgatgacttcttcaccccggtttataggtagtgcccctgctcggagagcccctgactgggttaaagtcacaggccccttgtctcaggtagagaccctgtccaggtaggacactttggctaaggttaaaagcttgttgaatcagtacaataactgatagtcgtggttgacacgcagacctcttacaagagtgtctaggtgcctttgagagttactctttgctctcttcggaagaacccttaggggttcgtgcatgggcttgcatagcaagtcttagaatgcgggtaccgtacagtgttgaaaaacactgtaaatctctaaaagagacca

SEQ ID NO: 4 - DNA, 2361 bp, Artificial Sequence
  Description of Artificial Sequence  Synthetic Primer
gtaaaataccaactaattctcgttcgattccggcgaacattctattttaccaacatcggttttttcagtagtgatactgtttttgttcccgatgtctaacatagtttctcccttcagtggttcctcacgaactacgtatgacgttggcaagcaagcgggaggtactagcgatgagaagctcattgagtcgctgttctctgaaaaggctgtgaaagagatagctgccgagtgtaaactcggatgttataactatctgaagtctaatgaaccccgcaactatatagacctggtgccaaagtcacacgtatctgcttggctctcatgggctacatccaagtatgataaaggagagttaccttccaggggattcatgaacgttccacgcatcgtttgttttctcgttcgtaccacagatagcgcagagtccggttctataaccgtgagcctgtgcgattctggtaaggctgctcgtgctggagtactcgaagccattgataatcaggaggccacaattcagttgtcggctttacctgctttgatagctttgacgcctagctatgattgtccgatggaagtcatcggcggtgatagcggtaggaatcgatgttttgggatagcaacccaacttagcggtgtggtggggacaacaggttccgttgcagttactcatgcgtattggcaagctaatttcaaagcgaagcccaacaactataagttgcatggtcccgctacaattatggtaatgccatttgacagactgagacaactcgataagaaaagcctcaaaaattatattagaggtatttctaaccagtctgtggatcatgggtatcttctcggaagaccgttacaatctgttgaccaggttgcccaggaagatttgttagttgaggaatccgagtctccttccgctctcggcagaggtgtgaaggatagtaagtctgtatccgcgtcatctgtcgctggacttcctgtgtccagtcctacgcttagagttaaataggtaaatccggtctaacaagctcggtccatttcgtagagttaagcaagctggggagacccccgacagccgtttggatcagcgctcgcgtctcgtttgggttcaattcccttaccttacaacggcgtgttgagataggtcctcgggggaggttatccatgtttgtggatattctatgttgtgtgtctgagttattattaaaaaaaaaaaaaaaaaaaagatctatgtcctaattcagcgtattaataatgtcgacttcaggaactggtaagatgactcgcgcgcagcgtcgtgctgccgctcgcagaaatcgttggaccgctagggtccaaccagtaattgtcgaaccactcgctgctggccaaggcaaggccattaaagcgattgcaggatacagcatatcaaagtgggaggcgtcttcggacgcgattacagcgaaagccaccaatgccatgagtatcactctgccccatgagctctcttctgaaaagaataaggagcttaaggtcggcagggtgctgctttggttgggacttcttcctagcgttgctgggaggattaaggcttgtgttgctgagaaacaggcacaggccgaggctgcttttcaagtagccttggcggttgcagactcctcgaaagaggtggtcgcggccatgtatacggacgcctttcgaggggcgactctgggggatttgcttaatctccagatttatctgtatgcatctgaagcagtgcctgctaaggcggtcgttgtacatctagaagttgagcacgtaaggcctacgttcgatgacttcttcaccccggtttataggtagtgcccctgctcggagagcccctgactgggttaaagtcacaggccccttgtctcaggtagagaccctgtccaggtaggacactttggctaaggttaaaagcttctcgagtgttcatatatggtttgacagaaaactgaacaacacatatgaccaccttcttttcagcaggagttcacttttaagtgtctatgcagacatgtcagtaacctgcaaggaatactatgacccaaaccgttcaatgctggagttggtctttgctcctgcagacgaatggattggtcgaagtgacactgaaatcatcgatgcaactatggaagagctagccaagttgtttccagatgaaagcttgttgaatcagtacaataactgatagtcgtggttgacacgcagacctcttacaagagtgtctaggtgcctttgagagttactctttgctctcttcggaagaacccttaggggttcgtgcatgggcttgcatagcaagtcttagaatgcgggtaccgtacagtgttgaaaaacactgtaaatctctaaaagagacca

SEQ ID NO: 5 - DNA, 20 bp, Artificial Sequence
  Description of Artificial Sequence  Synthetic Primer
gacaatggtctcttttagag

SEQ ID NO: 6 - DNA, 42 bp, Artificial Sequence
  Description of Artificial Sequence  Synthetic Primer
aattaaccctcactaaagggagagtagaccacggaacgaggt

SEQ ID NO: 7 - DNA, 38 bp, Artificial Sequence
  Description of Artificial Sequence  Synthetic Primer
aattaaccctcactaaagggagagtaaaccacggaacg

SEQ ID NO: 8 - DNA, 37 bp, Artificial Sequence
  Description of Artificial Sequence  Synthetic Primer
aattaaccctcactaaagggagagtaaaataccaact

SEQ ID NO: 9 - DNA, 31 bp, Artificial Sequence
  Description of Artificial Sequence  Synthetic Primer
cataagcttctcgagtgttcatatatggttt

SEQ ID NO: 10 - DNA, 29 bp, Artificial Sequence
  Description of Artificial Sequence  Synthetic Primer
cataagcttagacacttaaaagtgaactc

SEQ ID NO: 11 - DNA, 29 bp, Artificial Sequence
  Description of Artificial Sequence  Synthetic Primer
cataagctttcatctggaaacaacttggc

SEQ ID NO: 12 - DNA, 28 bp, Artificial Sequence
  Description of Artificial Sequence  Synthetic Primer
gagcattgacaagcgtgtgatcgagacg

SEQ ID NO: 13 - DNA, 30 bp, Artificial Sequence
  Description of Artificial Sequence  Synthetic Primer
ccctcaaacccagagatgggaacgaagggg

SEQ ID NO: 14 - DNA, 17 bp, Artificial Sequence
  Description of Artificial Sequence  Synthetic Primer
gttgggacttcttccta

SEQ ID NO: 15 - DNA, 2115 bp, Artificial Sequence
  Description of Artificial Sequence  Synthetic Primer
gtaaaataccaactaattctcgttcgattccggcgaacattctattttaccaacatcggttttttcagtagtgatactgtttttgttcccgatgtctaacatagtttctcccttcagtggttcctcacgaactacgtatgacgttggcaagcaagcgggaggtactagcgatgagaagctcattgagtcgctgttctctgaaaaggctgtgaaagagatagctgccgagtgtaaactcggatgttataactatctgaagtctaatgaaccccgcaactatatagacctggtgccaaagtcacacgtatctgcttggctctcatgggctacatccaagtatgataaaggagagttaccttccaggggattcatgaacgttccacgcatcgtttgttttctcgttcgtaccacagatagcgcagagtccggttctataaccgtgagcctgtgcgattctggtaaggctgctcgtgctggagtactcgaagccattgataatcaggaggccacaattcagttgtcggctttacctgctttgatagctttgacgcctagctatgattgtccgatggaagtcatcggcggtgatagcggtaggaatcgatgttttgggatagcaacccaacttagcggtgtggtggggacaacaggttccgttgcagttactcatgcgtattggcaagctaatttcaaagcgaagcccaacaactataagttgcatggtcccgctacaattatggtaatgccatttgacagactgagacaactcgataagaaaagcctcaaaaattatattagaggtatttctaaccagtctgtggatcatgggtatcttctcggaagaccgttacaatctgttgaccaggttgcccaggaagatttgttagttgaggaatccgagtctccttccgctctcggcagaggtgtgaaggatagtaagtctgtatccgcgtcatctgtcgctggacttcctgtgtccagtcctacgcttagagttaaataggtaaatccggtctaacaagctcggtccatttcgtagagttaagcaagctggggagacccccgacagccgtttggatcagcgctcgcgtctcgtttgggttcaattcccttaccttacaacggcgtgttgagataggtcctcgggggaggttatccatgtttgtggatattctatgttgtgtgtctgagttattattaaaaaaaaaaaaaaaaaaaagatctatgtcctaattcagcgtattaataatgtcgacttcaggaactggtaagatgactcgcgcgcagcgtcgtgctgccgctcgcagaaatcgttggaccgctagggtccaaccagtaattgtcgaaccactcgctgctggccaaggcaaggccattaaagcgattgcaggatacagcatatcaaagtgggaggcgtcttcggacgcgattacagcgaaagccaccaatgccatgagtatcactctgccccatgagctctcttctgaaaagaataaggagcttaaggtcggcagggtgctgctttggttgggacttcttcctagcgttgctgggaggattaaggcttgtgttgctgagaaacaggcacaggccgaggctgcttttcaagtagccttggcggttgcagactcctcgaaagaggtggtcgcggccatgtatacggacgcctttcgaggggcgactctgggggatttgcttaatctccagatttatctgtatgcatctgaagcagtgcctgctaaggcggtcgttgtacatctagaagttgagcacgtaaggcctacgttcgatgacttcttcaccccggtttataggtagtgcccctgctcggagagcccctgactgggttaaagtcacaggccccttgtctcaggtagagaccctgtccaggtaggacactttggctaaggttaaaagcttgttgaatcagtacaataactgatagtcgtggttgacacgcagacctcttacaagagtgtctaggtgcctttgagagttactctttgctctcttcggaagaacccttaggggttcgtgcatgggcttgcatagcaagtcttagaatgcgggtaccgtacagtgttgaaaaacactgtaaatctctaaaagagacca

SEQ ID NO: 16 - DNA, 29 bp, Artificial Sequence
  Description of Artificial Sequence  Synthetic Primer
cataagcttattatgagcaggagctggga

SEQ ID NO: 17 - DNA, 29 bp, Artificial Sequence
  Description of Artificial Sequence  Synthetic Primer
cataagctttctgctggaatgtgctgaga

SEQ ID NO: 18 - DNA, 29 bp, Artificial Sequence
  Description of Artificial Sequence  Synthetic Primer
cataagctttggagatggtgtcagtcaca

SEQ ID NO: 19 - DNA, 734 bp, Artificial Sequence
  Description of Artificial Sequence  Synthetic Primer
tcgatgacttcttcaccccggtttataggtagtgcccctgctcggagagcccctgactgggttaaagtcacaggccccttgtctcaggtagagaccctgtccaggtaggacactttggctaaggttaaaagctttctgctggaatgtgctgagagatgccaaaatagaaccaccaatccagacgctgtattttctttctggtggtgcaatcactttaaccttcatactgctaggagcaagggaagtgatctccttgctcatacgatcagcaattccaggaaacatggtagaccctccacttaggacaacattgccatacaggtccttccttatatctacatcacacttcatgatggagttgtatgtagcttcatgtatgccaggagattccataccaacaagagatggctggaacagaacctcagggcacctgaacctttctgacccaatggtaattacttggccatcaggcatctcatagcttttctcaacagaggagctgctcctagaagttcccagctcctgctcataataagcttgttgaatcagtacaataactgatagtcgtggttgacacgcagacctcttacaagagtgtctaggtgcctttgagagttactctttgctctcttcggaagaacccttaggggttcgtgcatgggcttgcatagcaagtcttagaatgcgggtaccgtacagtgttgaaaaacactgtaaatctctaaaagagacca